(19) United States
(12) Patent Application Publication
(10) Pub. No.: US 2026/0325014 A1
(43) Pub. Date: Oct. 22, 2026

(54) SYSTEMS, DEVICES, AND METHODS FOR MANAGING VEIN LOCATION

(71) Applicant: Sheila Kaitlyn Oliver, Mountain View, HI (US)

(72) Inventor: Sheila Kaitlyn Oliver, Mountain View, HI (US)

(21) Appl. No.: 19/722,688

(22) Filed: Jun. 26, 2026

Related U.S. Application Data
(63) Continuation-in-part of application No. 18/072,408, filed on Nov. 30, 2022, now Pat. No. 12,685,485.
(60) Provisional application No. 63/284,163, filed on Nov. 30, 2021.

(51) Int. Cl. A61B 5/15

(52) U.S. Cl. CPC A61B 5/1405

(57) ABSTRACT

The present disclosure provides a device for stabilizing a vein during venipuncture, blood withdrawal, medication injection, catheter placement, or other vascular access procedures. The device comprises a body having a C-shaped configuration with a first arm and a second arm separated by an open access gap. A first magnetic region is associated with the first arm, and a second magnetic region is associated with the second arm. The magnetic regions are arranged with like poles facing one another to generate a repulsive magnetic field across the access gap and into tissue beneath the device so as to stabilize a target vein aligned with the access gap. The opposing magnetic fields focus inwardly toward the target vein and provide bilateral stabilization of the vein during needle insertion while preserving a clear needle path through the access gap.

### FIELD OF INVENTION

The present disclosure relates generally to systems, devices, and methods for locating, stabilizing, and accessing veins, and more particularly to magnetic wearable or positionable devices that facilitate blood withdrawal, medication delivery, and related vascular access procedures.

### CROSS-REFERENCE TO RELATED APPLICATIONS

This application is a continuation-in-part of U.S. patent application Ser. No. 18/072,408, entitled “Systems, Devices, and/or Methods for Managing Vein Location”, filed on Nov. 30, 2022, which claims priority to U.S. Provisional Patent Application Ser. No. 63/284,163, entitled “Systems, Devices, and/or Methods for Managing Vein Location” filed on Nov. 30, 2021. The entire disclosure of each of the foregoing applications is incorporated herein by reference in its entirety for all purposes.

### BACKGROUND

Access to veins can be difficult in many patients because veins may be hard to visualize, may shift relative to surrounding tissue, and may roll laterally during needle insertion. Even where a vein is initially located, the act of depressing skin and advancing a needle can cause the target vessel to move, increasing the likelihood of multiple attempts, patient discomfort, bruising, and prolonged procedure time. These challenges can be even more significant in self-injection, home-use, or other one-handed procedures in which the user has reduced ability to manually tension tissue or stabilize the vessel while simultaneously manipulating a needle. Accordingly, there is a need for a device that can reliably stabilize a target vein while preserving a clear and unobstructed needle-access path, enabling safe and efficient venipuncture even in self-injection and one-handed use scenarios.

### SUMMARY

The following is a summary of the invention to provide a basic understanding of some aspects of the invention. This summary is not intended to identify key/critical elements of the invention or delineate the invention's scope. Its sole purpose is to present some concepts of the invention in a simplified form as a prelude to the more detailed description that is presented later.

The following is a summary of the invention to provide a basic understanding of some aspects of the invention. This summary is not intended to identify key/critical elements of the invention or delineate the invention's scope. Its sole purpose is to present some concepts of the invention in a simplified form as a prelude to the more detailed description that is presented later.

Certain exemplary embodiments provide systems, devices, and methods for managing vein location using magnetic structures, including magnetic bands, cuffs, and C-shaped or C-arc magnetic devices.

In various embodiments, provided herein is a device for stabilizing a vein of a user, comprising a C-shaped body defining an open needle-access gap; a first magnetic region disposed on a first arm of the C-shaped body; and a second magnetic region disposed on a second arm of the C-shaped body and spaced from the first magnetic region across the open needle-access gap, wherein like magnetic poles of the first magnetic region and the second magnetic region face each other to generate a repulsive magnetic field extending through a target region beneath skin of the user, and wherein the open needle-access gap is positioned to permit insertion of a needle into the vein while the repulsive magnetic field stabilizes the vein.

In many embodiments, the first magnetic region and the second magnetic region are oriented with like magnetic poles facing each other across the open needle-access gap so as to generate a repulsive magnetic field or force through the open needle-access gap. This configuration can produce opposing or bucking magnetic fields that focus inwardly through the open needle-access gap and into underlying tissue, thereby producing a tensioning, centering, and/or cradling effect on a vein without requiring a full annular obstruction around the intended puncture site.

In certain embodiments, the disclosed device comprises a body defining a C-shaped or C-arc geometry having a first arm and a second arm separated by an access gap. As the target vein is acted upon from opposing lateral sides of the first arm and the second arm, the vein may be less likely to roll sideways during needle insertion than with a one-sided magnetic arrangement. At the same time, because the device is open along the open needle-access gap, the configuration provides a clear needle path unavailable in a closed ring structure. Thus, the curved body due to the C-shaped or C-arc geometry can provide partial circumferential support and distributed pressure around the target area.

In certain embodiments, the disclosure provides a magnetic vein stabilizer configured to stabilize a vessel using opposing, tuned magnetic fields at a target micro-scale region. The magnetic fields may be tuned by selecting magnet strength, pole orientation, spacing, body geometry, or combinations thereof to hold the vessel steady during precise injection or venous access while reducing lateral rolling of the vessel.

In certain other embodiments, the C-shaped body is configured to focus the magnetic field inwardly toward the vein. In many embodiments, the C-shaped body has an arc length of approximately 100 mm, 150 mm, 200 mm, 220 mm, 230 mm, 240 mm, 250 mm, 260 mm, or 270 mm, an arc width of approximately 100 mm, 110 mm, 120 mm, 130 mm, 140 mm, or 150 mm, or a curvature radius of approximately 50 mm, 60 mm, 70 mm, 80 mm, 90 mm, or 100 mm. In many embodiments, the magnetic field forms a magnetic cradle that reduces the rolling movement of the vein during the insertion of the needle. In many embodiments, the open needle-access gap has a width of approximately 20 mm, 30 mm, 40 mm, or 50 mm.

In many embodiments, the magnetic field generated due to the positioning of the first magnetic region and the second magnetic region produces a localized magnetic field gradient beneath the skin of the user, wherein the localized magnetic field gradient can be concentrated at a depth corresponding to a position of the vein, and the magnetic field can laterally constrain the vein from opposing sides of the vein.

In certain embodiments, the magnetic field strength, spacing, geometry, pole orientation, and/or standoff distance are selected so that the device can stabilize the vein while reducing vein rolling, displacement, collapse, compression, or occlusion during access. In certain embodiments, the body shape and contact profile cooperate with the magnetic field to establish multiple spaced contact regions that hold surrounding tissue in a stable configuration while preserving accessibility through the open needle-access gap. In certain embodiments, the body is ergonomically contoured for placement against an arm curvature and configured for rapid removal after successful catheter, needle, or cannula placement.

In many embodiments, a spacing between the first magnetic region and the second magnetic region is selected to tune the location of the strongest portion of the magnetic field beneath the skin.

In certain embodiments, the device is wearable as a cuff, clip, band, patch, or arm-mountable structure. In certain embodiments, the device may include non-magnetic support materials such as polymers or metals, including rigid polymers, elastomeric materials, aluminum, copper, brass, stainless steel, adhesives, and/or hook-and-loop fasteners. In many embodiments, wherein at least one of the first magnetic region and the second magnetic region comprises Neodymium (Nd—Fe—B) or Samarium Cobalt (SmCo).

In various embodiments, the disclosed device is configured for removal after venipuncture, catheter placement, medication injection, or other vascular access procedure without substantially disturbing an inserted needle, catheter, cannula, or other access member. The open C-shaped configuration permits the device to be withdrawn laterally or lifted away from the target site while leaving the needle-access member in place, thereby reducing patient discomfort, limiting movement of the needle-access member, and minimizing procedure time.

In certain embodiments, the device includes an optimized tissue-contact surface configured to distribute contact pressure around a target insertion region.

In many embodiments, the disclosure provides a method of using a device for stabilizing a vein of a user, comprising: positioning the device over tissue of the user, the device comprising a C-shaped or C-arc body having a first magnetic region and a second magnetic region separated by an aperture or an open needle-access gap and oriented with like magnetic poles facing each other; aligning the aperture or the open needle-access gap with a target vein of the user; magnetically stabilizing the target vein with a repulsive magnetic field extending across the aperture or open needle-access gap and into tissue beneath the aperture or open needle-access gap; inserting a needle through the aperture or open needle-access gap and into the target vein; and removing the device from the tissue by lifting the C-shaped or C-arc body away from the tissue surface or sliding it laterally relative to the inserted needle, without substantially disturbing or repositioning the needle, catheter, cannula, or other access member.

In many embodiments, the disclosure provides a method of stabilizing a vein of a user, comprising positioning a C-shaped magnetic device adjacent to skin of the user, the C-shaped magnetic device defining an open needle-access gap and comprising a first magnetic region and a second magnetic region spaced apart across the open needle-access gap with like magnetic poles facing each other; generating, by the first magnetic region and the second magnetic region, a repulsive magnetic field extending through a target region beneath the skin; stabilizing the vein with the repulsive magnetic field; and inserting a needle through the open needle-access gap and into the vein while the vein is stabilized by the repulsive magnetic field. In many embodiments, the method further comprises maintaining spaced contact points around a target insertion area while inserting the needle and withdrawing a blood sample from the vein through the needle or injecting a medication into the vein through the needle.

In certain embodiments, methods are provided for placing the device over a portion of a limb, aligning the open needle-access region over a target vein, establishing contact between the device and the tissue, magnetically stabilizing the vein, verifying vein visibility and/or accessibility through the access region, and inserting a needle through the needle-access region to withdraw blood and/or deliver a medication. In certain embodiments, the device is maintained in position during venipuncture and is removed after successful catheter placement or completion of the access procedure.

In certain embodiments, the spacing between the first magnetic region and the second magnetic region is adjustable and the strength of the magnetic field is calibrated to stabilize the vein while reducing rolling, displacement, collapse, compression, or occlusion that could interfere with blood flow through the vein.

In many embodiments, the disclosed method provides a system for stabilizing a vein of a user during self-injection, comprising a wearable support configured to be positioned adjacent skin of the user; a C-shaped magnetic stabilizer coupled to the wearable support, the C-shaped magnetic stabilizer defining an open needle-access gap; a first magnetic region disposed on a first side of the open needle-access gap; and a second magnetic region disposed on a second side of the open needle-access gap with like magnetic poles of the first magnetic region and the second magnetic region facing each other, wherein the first magnetic region and the second magnetic region are configured to generate a repulsive magnetic field that laterally stabilizes the vein while the open needle-access gap permits insertion of a needle into the vein, and wherein the system is configured for single-handed placement or adjustment by the user.

These and other features, aspects, and advantages of the present systems and methods will become better understood with references to the following figures and descriptions. This summary is an introduction to the concepts. Additional aspects and advantages of this invention will be apparent from the following detailed description of preferred embodiments, which proceeds with reference to the accompanying drawings.

### BRIEF DESCRIPTION OF DRAWINGS

The accompanying drawings, where like reference numerals refer to identical or functionally similar elements throughout the separate views, together with the detailed description below, are incorporated in and form part of the specification and serve to illustrate further embodiments of concepts that include the claimed disclosure, and explain various advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 is a perspective view of an exemplary embodiment of a magnetic band 1000.

FIG. 2 is a plan view of an exemplary embodiment of a band magnetic band 2000.

FIG. 3 is a plan view of an exemplary embodiment of a magnetic band 3000.

FIG. 4 is a flowchart of an exemplary embodiment of a method 4000.

FIG. 5 is a perspective view of an exemplary embodiment of a system 5000.

FIG. 6 is a perspective view of an exemplary embodiment of a device 6000.

FIG. 7 is a schematic conceptual view of an exemplary labeled magnetic vein stabilizer 7000.

### DETAILED DESCRIPTION

While the presently disclosed systems and methods are susceptible to embodiment in many different forms, there is shown in the drawings and will herein be described in detail several specific embodiments with the understanding that the present disclosure is to be considered as an exemplification of the principles of the presently disclosed systems and methods and is not intended to limit the disclosure systems and methods to the embodiments illustrated.

Exemplary embodiments of the methods/systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the disclosure warranted hereon are all such embodiments that reasonably fall within the scope of the advancement to the art hereby contributed.

Certain exemplary embodiments of the disclosure provide a device for managing and stabilizing vein location and needle access. The device can be configured to assist a clinician or user in identifying, stabilizing, and accessing a vein for blood withdrawal, medication injection, catheter placement, or related procedures.

Certain exemplary embodiments provide a device comprising a body, such as a magnetic body, configured to locate, stabilize, and/or access a vein of a user. In certain embodiments, the magnetic body comprises a band, cuff, or C-shaped or c-arc structure having a needle-access region through which a needle can be inserted into the vein to withdraw blood and/or deliver medication. In certain embodiments, the structure comprises a first arm and a second arm separated by an open needle-access gap, wherein the first arm and the second arm are each configured to carry a respective magnetic region. In certain embodiments, opposed magnetic regions are arranged so that a magnetic field extends across the needle-access region and into tissue beneath the device to stabilize the vein, optionally with like poles facing one another (e.g., a north pole facing a north pole or a south pole facing a south pole) to generate a repulsive magnetic field. Thus, the method can utilize the magnetic body to restrain the motion of the vein while blood is taken from the vein or a medication is injected into the vein.

The present disclosure expands further from U.S. patent application Ser. No. 18/072,408, entitled “Systems, Devices, and/or Methods for Managing Vein Location”, filed on Nov. 30, 2022, the disclosure of which is incorporated herein in entirety, wherein the disclosure herein describes a C-shaped or c-arc structure that improves lateral stabilization while preserving access to the target vein.

Certain exemplary embodiments provide a device comprising a band comprising a magnetic material. Certain exemplary embodiments provide a method comprising utilizing a magnetic band to restrain the motion of a vein in a human, wherein a blood sample is taken from the vein, or a medication is injected into the vein.

Certain exemplary embodiments provide a band that is wearable by a human (for example, a band worn on the wrist of a user) that can allow blood veins to be located and restrained from the motion such that blood can be drawn from the human more easily and/or more effectively. In certain embodiments, application of a magnetic field may change or help maintain vein position and may support blood flow by reducing vein rolling, displacement, collapse, compression, or occlusion during access. When a magnetic field is applied, veins can be easier to see and/or easier to insert a needle in for drawing blood and/or injecting medications. Magnetic fields can restrain the motion of a vein once the skin is depressed in order to cause a needle to enter the vein. Via the restraint of motion by a magnetic field, insertion of a needle can be easier and/or fewer unsuccessful attempts to insert the needle can be possible.

At least one study has shown a correlation between an application of magnetic fields to blood vessel size. “Rotating magnetic field delays human umbilical vein endothelial cell aging and prolongs the lifespan Caenorhabditis elegans”, Xu J, Liu K, Chen T, Zhan T, Ouyang Z, Wang Y, Liu W, Zhang X, Sun Y, Xu G, Wang X, Aging (Albany NY). 2019 Nov. 22; 11(22):10385 10408. doi: 10.18632/aging.102466. Epub 2019 Nov. 22. PMID: 31757933; PMCID: PMC6914427. Applicant hereby incorporates the article “Rotating magnetic field delays human umbilical vein endothelial cell aging and prolongs the lifespan of Caenorhabditis elegans” by reference in its entirety.

A magnetic field can also facilitate the positioning of a tourniquet to restrict blood flow.

Oxygenated hemoglobin molecules aren't attracted to a strong magnetic field because such molecules are diamagnetic. This means oxygenated hemoglobin molecules are weakly repelled by a magnetic field due to a lack of unpaired electrons. Hemoglobin molecules that haven't been oxygenated, on the other hand, have four unpaired electrons, making them slightly paramagnetic, or weakly attracted to a magnetic field. Because most of the blood in human bodies is made up of water (which is also diamagnetic) and oxygenated hemoglobin, human blood is, overall, diamagnetic, and therefore subtly repelled by magnetic fields. This property can be utilized for improved devices, systems, and/or methods for drawing blood and/or injecting medications.

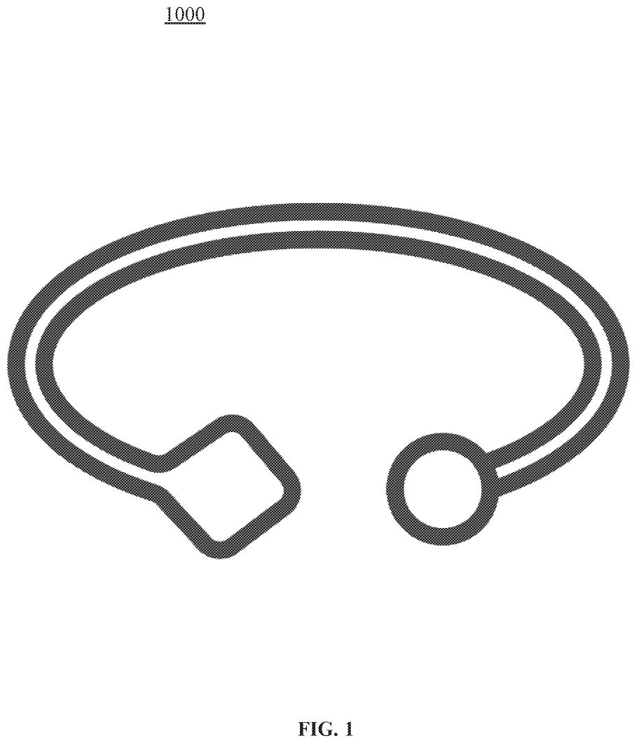

FIG. 1 is a perspective view of an exemplary embodiment of a band 1000, which illustrates an open-ended band with shaped ends. Other band shapes are possible. For example, different end shapes can be used. In addition, any cross-sectional shape can be utilized. The illustrated embodiment has a substantially circular cross-section. Other embodiments can have a bevelled or other cross-section. Band 1000 comprises a magnetic material such as a rare earth magnet material.

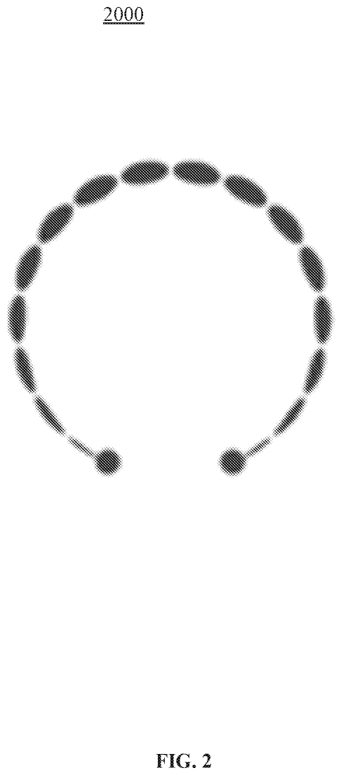

FIG. 2 is a plan view of an exemplary embodiment of a band 2000, which illustrates a coupled set of magnetic links. Certain users might prefer the aesthetics of Band 2000 relative to other possible appearances.

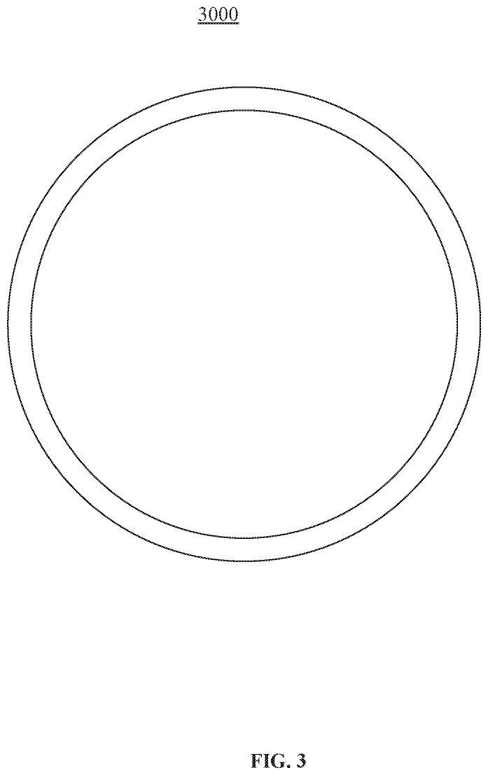

FIG. 3 is a plan view of an exemplary embodiment of a band 3000, which illustrates an annular shape.

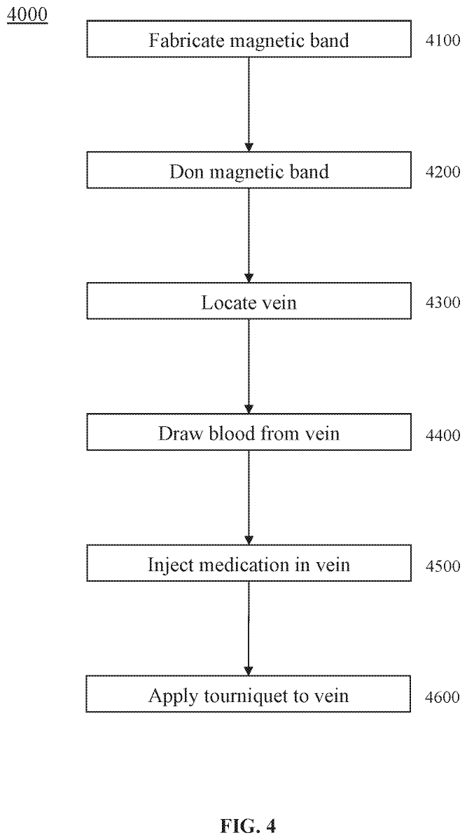

FIG. 4 is a flowchart of an exemplary embodiment of a method 4000. At activity 4100, a magnetic band can be fabricated. At activity 4200, a user can don the magnetic band. At activity 4300, a vein can be located while the user is wearing the magnetic band. At activity 4400, blood can be drawn from the vein. At activity 4500, a medication can be injected into the vein. At activity 4600, a tourniquet can be applied to the vein.

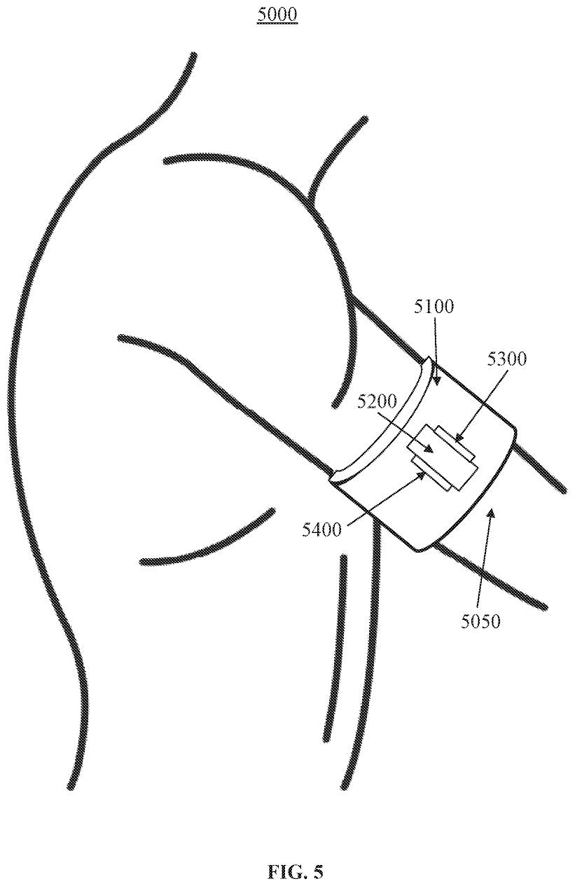

FIG. 5 is a perspective view of an exemplary embodiment of a system 5000, which comprises a cuff 5100 that is coupled to a human forearm 5050. Cuff 5100 defines an aperture 5200. A user can insert a needle into the human forearm 5050 via aperture 5200. Cuff 5100 comprises a first magnet 5300 and a second magnet 5400. First magnet 5300 and second magnet 5400 create tension on one or more veins in the human forearm 5050. In certain exemplary embodiments, first magnet 5300 and second magnet 5400 are oriented with like magnet poles facing one another across aperture 5200 to generate a repulsive magnetic field extending between first magnet 5300 and second magnet 5400 and through aperture 5200 into soft tissue of human forearm 5050, thereby creating tension on one or more veins in human forearm 5050.

Materials utilized for cuff 5100 can comprise copper, aluminum, brass, and/or stainless steel, etc.

Introducing a C-shaped or C-arc magnetic structure can provide bilateral stabilization of a target vein while maintaining an open needle-access gap. Further, the C-arc can conform to local anatomy and support intuitive single-handed placement and removal.

The disclosed device may be in the form of a band, open-ended band, cuff, or related wearable body configured for placement on a wrist, forearm, arm, or other body region. The body may be rigid, semi-rigid, flexible, elastic, adjustable, and/or attachable by fasteners or adhesives. The device may be adjustable in size; secured by hook-and-loop fasteners; adhered to skin; strapped to a limb; clipped onto tissue or a support band; incorporated into a wearable cuff, and/or hand-held and temporarily positioned during access. The disclosed device can be ergonomically positioned with one hand and removed after the vascular access procedure with minimal disturbance to an inserted needle or catheter.

The device comprises a magnetic material; the magnetic material comprises Neodymium (“Neodymium” or “Nd—Fe—B”, referring to neodymium-iron-boron magnetic alloy) and/or Samarium Cobalt (SmCo); the device is worn for a period prior to venous access; the device is usable with non-magnetic stainless steel needles; the device can be used for blood withdrawal, medication injection, and/or other vascular access procedures.

In certain exemplary embodiments, a device comprises a C-shaped body or C-arc body having: a first arm; a second arm; a curved connecting portion joining the first arm and second arm; and an open needle-access gap between the free ends of the first and second arms, the open needle-access gap being alignable with an insertion site over a target vein.

The body can be sized and shaped for placement over a forearm, wrist, hand, antecubital region, or other body location where venous access is desired. The body may be a complete wearable cuff, a partially wrapping support, a local positioning frame, a hand-held instrument, or an adhesive-backed placement member. In certain embodiments, the body is curved to conform naturally to an arm cross-section or other local anatomy so that the device can be positioned intuitively, including with one hand.

In certain embodiments, the c-arc body is substantially planar; arcuate in top view; contoured to match local anatomy; open-ended for easy placement and removal; and/or configured to position the access gap directly over a target vein and corresponding insertion site.

The present disclosure provides a device with a magnetic body with a first magnet and a second magnet, wherein the first magnetic region can be disposed on, in, or adjacent the first arm, and the second magnetic region can be disposed on, in, or adjacent the second arm. Each magnetic region may comprise one magnet, a stack of magnets, a magnetic insert, a magnetic composite, a magnet wrapped with conductive material, or a manufactured magnetic assembly.

The first and second magnetic regions are laterally opposed across the needle-access gap and spaced apart so that a target vein lies generally between them when the device is properly positioned, optionally within a stabilized zone beneath the needle-access gap.

In certain embodiments, the first and second magnetic regions are oriented with: like poles facing one another across the needle-access gap to generate a repulsive magnetic field; selected pole orientation to shape the field through the needle-access gap and underlying tissue; and/or a field distribution that creates a stabilization zone beneath the needle-access gap, including a magnetic cradle or laterally constraining zone for the vein.

A principal advantage of the c-arc geometry is that it can act on the target vessel from both lateral sides, reducing or resisting side-to-side vein rolling during skin depression and needle advancement. Because the device remains open along the needle-access gap, a clinician may approach the target vein directly through the open region without substantial obstruction from overlying device structure.

The combination of bilateral stabilization and an unobstructed central approach can create a preferred needle access path, insertion region, or sweet spot for venipuncture. In certain embodiments, the sweet spot corresponds to a needle access path through the open needle-access gap, where the vein may remain visible and accessible through the opening while surrounding tissue is held in a stable configuration.

In certain embodiments, the disclosed device is configured so that the magnetic field strength is calibrated, tuned, or otherwise selected to stabilize the vein while reducing rolling, displacement, collapse, compression, or occlusion that could interfere with blood flow. This can be achieved by selecting one or more of: magnet strength; magnet material; magnet dimensions; pole orientation; spacing between the magnetic regions, including pole-gap spacing across the access gap; spacing between the device and the skin; geometry and/or depth of the c-arc body; local support contour; cushioning, interface layers, and/or contact-point configuration; and/or duration of placement.

In some embodiments, the device may be described as: a C-shaped cuff; an open magnetic frame; a partial annulus; an open arcuate magnet support; or an aperture-defining support body in which the open region is not fully enclosed.

In certain embodiments, a method of using a device for stabilization of a vein comprises providing a C-arc magnetic device; positioning the device on or adjacent tissue overlying a target vein area; aligning the needle-access gap with the target vein and an insertion site; allowing the magnetic field to stabilize or tension the vein; inserting a needle through the needle-access gap and into the stabilized vein while maintaining the position of the device; withdrawing blood, injecting medication, placing a catheter, and/or performing another vascular access procedure; and removing the device from the tissue by lifting the C-shaped or C-arc body away from the tissue surface or sliding it laterally relative to the inserted needle, without substantially disturbing or repositioning the needle, catheter, cannula, or other access member.

The device is worn for a short time prior to needle insertion and is adjusted until the clinician identifies a preferred insertion location. In certain embodiments, the needle-access gap is centered over the visually identified or palpated vein, and the clinician can verify that the vein remains visible and accessible through the needle-access gap before puncture, with the device being positioned or adjusted with one hand. The C-shaped or C-arc body is further configured to facilitate quick removal after successful vascular access has been achieved, allowing the body to be disengaged from the target region via the open needle-access gap while the inserted access member remains in place.

The device can utilize any suitable materials and configurations consistent with the teachings of the parent disclosure and the new C-arc improvement.

In certain embodiments, the magnetic regions comprise Neodymium (“Neodymium” or “Nd—Fe—B”, referring to neodymium-iron-boron magnetic alloy) and/or Samarium Cobalt (SmCo); other rare-earth magnetic materials; magnetic composites; and/or multi-part magnet structures.

In certain exemplary embodiments, each magnetic region comprises one or more cylindrical Neodymium N42 grade magnets. In one non-limiting embodiment, each magnet has a diameter of approximately 5-10 mm, and a thickness or length of approximately 1-3 mm. In certain embodiments, such magnets have a surface magnetic field strength of approximately 3,400 Gauss.

The foregoing values are exemplary and non-limiting. Magnet grade, magnet size, number of magnets, pole orientation, spacing, separation distance, field strength, repulsive force, and manufacturing tolerances may be varied to achieve effective stabilization of the target vein while reducing excessive tissue compression, vein collapse, venous occlusion, discomfort, or interference with surrounding tissue or medical equipment.

The support body or housing can comprise: rigid polymer; semi-rigid polymer; elastomeric material; aramid material, including Kevlar; copper; aluminum; brass; stainless steel; laminated materials; and/or skin-compatible adhesive layers.

In certain embodiments, magnets are wrapped with material such as copper, or conductive material is incorporated into a magnetic assembly. In certain embodiments, the body further includes a skin-contact interface, pad, or cushioning layer configured to distribute pressure during placement.

In certain embodiments, the support body, housing, skin-contact interface, pad, or cushioning layer comprises medical-grade silicone, thermoplastic elastomer (“TPE”), medical-grade polyurethane, or combinations thereof. Medical-grade silicone may provide biocompatibility, flexibility, patient comfort, and ease of sterilization. TPE may provide a soft-touch skin-contact surface, chemical resistance, and elastic compliance. Medical-grade polyurethane may provide durability with controlled flexibility, abrasion resistance, and suitability for repeated placement and removal during vascular access procedures.

In certain embodiments, the free ends of the arms are shaped to define the needle-access gap and to optimize visibility, accessibility, and maneuverability of the needle.

This configuration can reduce disturbance of the inserted access member, may decrease patient discomfort, and may shorten the time required to complete a venipuncture, intravenous catheter placement, medication injection, or blood withdrawal procedure. In certain embodiments, one or more rounded edges, flexible portions, release tabs, low-friction surfaces, adhesive-release features, or separable body portions may further assist removal of the device from the patient after the access procedure.

In certain embodiments, the C-shaped or C-arc body includes one or more tissue-contact surfaces configured to distribute pressure over a selected contact area adjacent the target vein. The contact surfaces may be shaped, contoured, widened, rounded, cushioned, or otherwise configured to reduce localized pressure concentrations that could indent the skin or displace the vein. In some embodiments, the contact surfaces are arranged around, rather than directly over, the intended needle insertion site so that the open needle-access gap remains available for needle entry.

The distributed pressure profile can cooperate with the magnetic field to stabilize the vein while avoiding excessive mechanical compression of the tissue. In certain embodiments, the device maintains sufficient contact with the skin to resist movement of the device and reduce rolling or shifting of the vein, while limiting indentation of the vein. The contact surface area, curvature, material compliance, cushioning, and spacing between contact regions may be selected to balance patient comfort with effective vein stabilization.

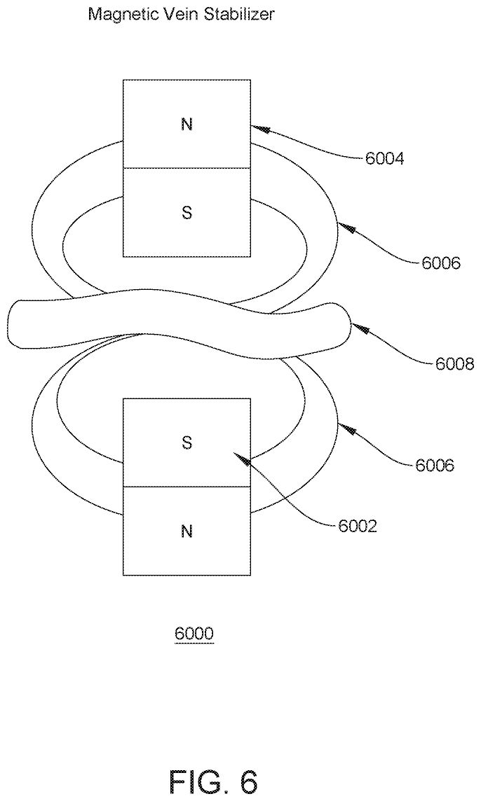

FIG. 6 illustrates an exemplary magnetic needle-access device 6000. Device 6000 may include a first or top magnet or magnetic region 6004, a second or bottom magnet or magnetic region 6002, magnetic field lines 6006 extending between or from the magnetic regions, and a central wavy element representing a target vein or vessel 6008 positioned between the magnetic regions. In certain embodiments, magnetic field lines 6006 may pass through or be adjacent to the target vein or vessel 6008 to define an access or stabilization region. The first magnetic region 6004 and second magnetic region 6002 may have selectable pole orientations, including a preferred like-pole orientation that generates a repulsive magnetic field or magnetic resistance through the access or stabilization region, without limiting the device to attraction between opposite poles.

In certain embodiments, the first magnetic region 6004 and second magnetic region 6002 are arranged so that like poles face across the access or stabilization region, causing a repulsive field, field gradient, or magnetic resistance to extend through the access or stabilization region and into tissue. The resulting magnetic field lines 6006 can stabilize a target vein or vessel 6008 against lateral rolling, displacement, collapse, compression, or occlusion and, in certain embodiments, may support blood flow through the vein or vessel 6008 while preserving an unobstructed insertion path for a needle or other access tool.

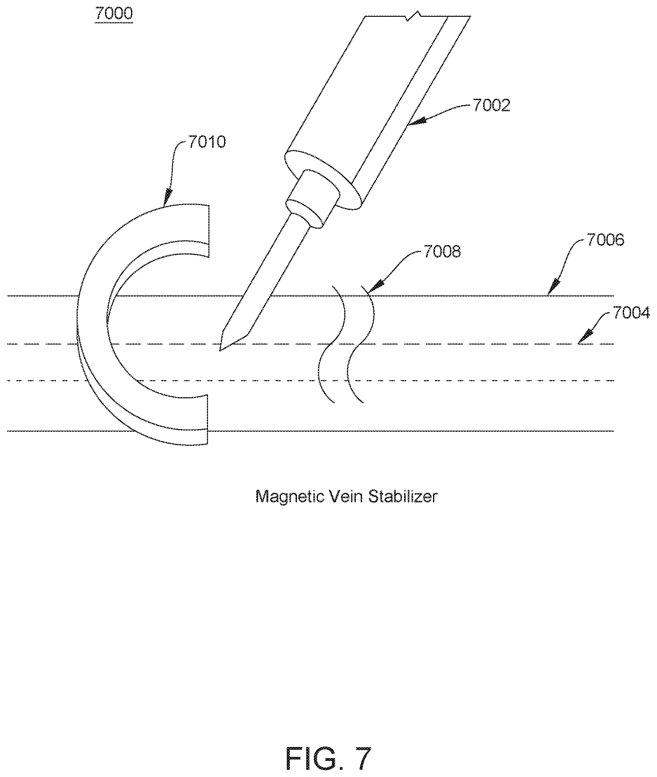

In certain embodiments, as illustrated schematically in FIG. 7, a magnetic vein stabilizer 7000 includes a needle 7002, cannula, catheter introducer, or other tool advanced toward an access region, dashed or boundary lines 7004 indicating a target region or boundary, a vessel, vein, or tissue reference line 7006, field or stabilization lines 7008, and a curved C-shaped, U-shaped, or arc-shaped stabilizer component 7010 positioned relative to the access region. The magnetic fields may be selected, oriented, or tuned, including by a like-pole repulsive orientation of magnetic regions where desired, to help hold a vein steady during needle insertion, medication injection, blood withdrawal, catheter placement, or other vascular access procedure. In certain embodiments, the magnetic field strength, spacing between magnetic regions, pole orientation, and geometry of the C-shaped, U-shaped, or arc-shaped component are selected to stabilize the vein while preserving an open access path for the needle or tool.

The disclosure also encompasses embodiments in which the C-arc device is used on veins located in the forearm, wrist, hand, arm, or other accessible vascular region.

When the following terms are used substantively herein, the accompanying definitions apply. These terms and definitions are presented without prejudice, and, consistent with the application, the right to redefine these terms during the prosecution of this application or any application claiming priority hereto is reserved. For the purpose of interpreting a claim of any patent that claims priority hereto, each definition (or redefined term if an original definition was amended during the prosecution of that patent), functions as a clear and unambiguous disavowal of the subject matter outside of that definition.
band—a circle of material. device—a machine, manufacture, and/or collection thereof.
Kevlar—Poly-paraphenylene terephthalamide, which is a strong, heat-resistant synthetic fiber, related to other aramids.
magnetic material—a substance or object that produces a magnetic field.
medication—a substance used in treating disease or relieving pain.
method—a process, procedure, and/or collection of related activities for accomplishing something.
motion—a process via which something changes position from one location to another.
system—a collection of mechanisms, devices, machines, articles of manufacture, processes, data, and/or instructions, the collection designed to perform one or more specific functions.
tourniquet—a device constructed for stopping the flow of blood through a vein or artery, typically by compressing a limb with a cord or tight bandage.
vein—any of a plurality of tubular branching vessels that carry blood from capillaries toward a heart of a human.
wearer—a human that dons something.

Exemplary embodiments of the methods/systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed and that that scope shall not be restricted, except in the light of the appended claims and their equivalents.

The foregoing description of the implementations of the present techniques and technologies has been presented for the purposes of illustration and description. It is not intended to be exhaustive or to limit the present techniques and technologies to the precise form disclosed. Many modifications and variations are possible in light of the above teaching. It is intended that the scope of the present techniques and technologies be limited not by this detailed description. The present techniques and technologies may be implemented in other specific forms without departing from the spirit or essential characteristics thereof. Accordingly, the disclosure of the present techniques and technologies is intended to be illustrative but not limiting.

What is claimed is:

1. A device for stabilizing a vein of a user, comprising:
a C-shaped magnetic body defining an open needle-access gap;
a first magnetic region disposed on a first arm of the C-shaped magnetic body; and
a second magnetic region disposed on a second arm of the C-shaped magnetic body and spaced from the first magnetic region across the open needle-access gap, wherein the first magnetic region and the second magnetic region comprise like magnetic pole faces facing one another across the open needle-access gap and the first magnetic region and the second magnetic region are configured to generate a repulsive magnetic field extending through the open needle-access gap, and
wherein the open needle-access gap is positioned to permit insertion of a needle into the vein while the repulsive magnetic field stabilizes the vein during insertion of the needle.

2. The device of claim 1, wherein the C-shaped magnetic body is configured to focus the repulsive magnetic field inwardly toward the vein.

3. The device of claim 1, wherein the repulsive magnetic field comprises magnetic field lines or a localized magnetic field gradient that produces magnetic resistance through the open needle-access gap beneath the skin of the user.

4. The device of claim 1, wherein the repulsive magnetic field laterally constrains the vein from opposing sides of the vein.

5. The device of claim 1, wherein the repulsive magnetic field forms a magnetic cradle that stabilizes a position of the vein and reduces rolling movement of the vein during insertion of the needle.

6. The device of claim 1, wherein at least one of the first magnetic region and the second magnetic region comprises Neodymium (Nd—Fe—B) or Samarium Cobalt (SmCo).

7. A method of stabilizing a vein of a user, comprising:
positioning a C-shaped magnetic device adjacent the skin of the user, the C-shaped magnetic device defining an open needle-access gap and comprising a first magnetic region and a second magnetic region spaced apart across the open needle-access gap with like magnetic pole faces facing one another;
generating, by the first magnetic region and the second magnetic region, a repulsive magnetic field extending through a target region beneath the skin;
stabilizing the vein with the repulsive magnetic field; and
inserting a needle through the open needle-access gap and into the vein while the vein is stabilized by the repulsive magnetic field.

8. The method of claim 7, wherein the C-shaped magnetic device is configured to focus the magnetic field inwardly toward the vein.

9. The method of claim 7, wherein the repulsive magnetic field comprises magnetic field lines or a localized magnetic field gradient extending through the open needle-access gap.

10. The method of claim 7, further comprising withdrawing a blood sample from the vein through the needle or injecting a medication into the vein through the needle.

11. The method of claim 7, wherein at least one of the first magnetic region and the second magnetic region comprises Neodymium (Nd—Fe—B) or Samarium Cobalt (SmCo).

12. The method of claim 7, wherein the repulsive magnetic field forms a magnetic cradle that stabilizes a position of the vein and reduces rolling movement of the vein during insertion of the needle.

13. A system for stabilizing a vein of a user during self-injection, comprising:
a wearable support configured to be positioned adjacent the skin of the user;
a C-shaped magnetic stabilizer coupled to the wearable support, the C-shaped magnetic stabilizer defining an open needle-access gap;
a first magnetic region disposed on a first side of the open needle-access gap; and
a second magnetic region disposed on a second side of the open needle-access gap with like magnetic pole faces of the first magnetic region and the second magnetic region facing one another,
wherein the first magnetic region and the second magnetic region are configured to generate a repulsive magnetic field that laterally stabilizes the vein during insertion of a needle into the vein through the open needle-access gap, and wherein the system is configured for single-handed placement or adjustment by the user during self-injection.

14. The system of claim 13, wherein the repulsive magnetic field extends through the open needle-access gap and into tissue beneath the open needle-access gap.

15. The system of claim 13, wherein the C-shaped magnetic stabilizer is configured to focus the magnetic field inwardly toward the vein.

16. The system of claim 13, wherein the repulsive magnetic field forms a magnetic cradle that stabilizes a position of the vein and reduces rolling movement of the vein during insertion of the needle.

17. The system of claim 13, wherein the system is configured to stabilize the vein during self-injection without requiring external compression of tissue adjacent to the vein by a second hand of the user.
